(19) United States
(12) Patent Application Publication
(10) Pub. No.: US 2008/0216756 A1
(43) Pub. Date: Sep. 11, 2008

(54) Method Of Cultivating Animals To Develop A Desired Color And To Increase Their Rate Of Growth

(75) Inventor: Frank L. Horsfall, Mont-sur-Rolle (CH)

Correspondence Address: MORGAN LEWIS & BOCKIUS LLP, 1111 PENNSYLVANIA AVENUE NW, WASHINGTON, DC, 20004, US

(73) Assignee: COHO HYDROCLEAN S.A., Nyon (CH)

(21) Appl. No.: 11/664,738

(22) PCT Filed: Oct. 6, 2005
(86) PCT No.: PCT/US2005/035796

§ 371 Date: Dec. 21, 2007

Related U.S. Application Data
(60) Provisional application No. 60/616,645, filed on Oct. 8, 2004.

(51) Int. Cl. A01K 61/00; C12N 1/20; C12N 1/00

(52) U.S. Cl. 119/204; 426/61; 426/7; 119/200; 119/215

(57) ABSTRACT

The present invention provides an animal feed consisting of photosynthetic bacteria which is useful for imparting desired color to an animal and for increasing the growth rate of the animal. Specifically, the feed is given to farm raised aquatic animals such as salmon so that they develop a more palatable pink flesh.

### RELATED APPLICATION

This application claims the benefit of priority of U.S. Provisional Application 60/616,645, which is incorporated by reference in its entirety.

### FIELD OF THE INVENTION

The present invention relates to photosynthetic bacteria as feed additive for animals, including aquatic animals.

### BACKGROUND OF THE INVENTION

Body coloration plays important roles in health and development as well as survival of animals and plants. Chromatophores in plants and animals contain pigments that impart color to the tissues or cells of animals and plants. One of the most common groups of naturally occurring pigments found in animals and plants is the carotenoids. Over 600 carotenoids have been identified in nature. Carotenoids are lipid soluble, dominate in giving yellow, orange, red, or purple color.

Carotenoids have long been recognized to be important as vitamin A precursors, components of chromatophores, immunological enhancers, visual pigments and as antioxidants. These pigments are also important in the coloration of aquatic animals such as salmon. In fact, it has been shown that these pigments have a significant impact on the sensory qualities of seafood in the marketplace. As an example, the distinctive pink color is important to the marketability of salmon. However, salmon cannot endogenously synthesize these pigments.

Salmon growing in the wild have flesh that is naturally pink in color because they consume crustaceans such as shrimp and other organisms rich in a carotenoid compound known as astaxanthine. Astaxanthine is found in the eyes and the carapace of the crustaceans.

Raised in captivity, salmon flesh is not colored, and therefore not saleable as such. Farm raised salmon are pale in color due to the lack of the color enhancement pigments. To impart color to the flesh of farm raised salmon, chemically produced astaxanthine is added to the feed given to the farm raised salmon. This process of feeding salmon chemically produced astaxanthine renders the meat saleable and visually acceptable to the public.

There are two primary commercial sources for astaxanthine. Astaxanthine can be obtained by extraction from crustacean shells or by chemical synthesis. Examples of processes for extraction of astaxanthine from crustacean shell and tissue waste are described (see U.S. Pat. Nos. 3,906,112 and 4,505,936; Journal of Food Science, Volume 47 (1982)). For example, whole crawfish waste is ground-up and admixed with water; the pH of the solution is adjusted with an alkali or acid; an enzyme is added to the solution; and the solution stirred, heated and hydrolyzed. After hydrolysis, the astaxanthine is extracted with oil, and the astaxanthine enriched oil is recovered by centrifugation. However, the cost of natural isolates of astaxanthine, especially from krill and crawfish shells, can cost anywhere from $5,000 to $15,000 per kilogram. Obviously, a less source dependent and more economical process for production of astaxanthine is needed.

Pigmentation of salmon has also been accomplished using synthetic carotenoid canthaxanthine as a feed additive, but this chemical is rather expensive and has been reported to produce a somewhat unsatisfactory color in salmon. Recent work in chemical synthesis of astaxanthine is exemplified in U.S. Pat. Nos. 4,245,109, 4,283,559, and 4,585,885. The present cost of synthetic astaxanthine pigment is approximately $2,000 per kilogram. Many countries, however, prohibit the use of synthetic carotenoids.

Although astaxanthine has been approved by the U.S. Food and Drug Administration to be incorporated into salmon diets, recently, there has been increasing concern over health risks associated with the addition of chemical additives to fish feed, such as astaxanthine. In the EU, the allowable concentration of astaxanthine in fish food has been reduced substantially. In the U.S., there is a ban on various synthetic coloring agents which have a potential for carcinogenicity and/or teratogenicity. Yellow and red azo dyes which are increasingly prohibited from use in foods are being replaced with non-toxic carotenoids. Although carotenoids are generally not toxic even at high levels, naturally occurring carotenoids are the preferred pigment for coloring salmon.

### SUMMARY OF THE INVENTION

The present invention provides a composition comprising microorganisms that produce carotenoids and animal feed. Also, the present invention provides animal feed comprising microorganisms. Moreover, the present invention discloses a method of making an animal feed comprising growing the microorganism, harvesting the microorganism, and adding the microorganism to an animal feed.

The present invention also discloses a method of cultivating an animal to develop a desired color or pigment comprising obtaining an animal feed supplemented with an effective amount of a microorganism, administering the animal feed to the animal, and cultivating the animal to grow under conditions that allow development of the color or pigment. Further, the present invention provides a method of cultivating an animal to develop an enhanced growth rate by feeding the animal with the animal feed supplemented with an effective amount of a microorganism. If only the increase in growth rate is desired, the microorganism may be grown under aerobic conditions. Additionally, the present invention discloses a method of decreasing the amount of waste excretion by an animal comprising feeding the animal with the animal feed supplemented with an effective amount of a microorganism.

The microorganisms used in the present invention produce carotenoids. In one embodiment, the carotenoids are xanthophylls. In a preferred embodiment, the xanthophylls are astaxanthines.

In one embodiment, the microorganism is a bacterium. Preferably, the bacterium is the photosynthetic purple non-sulfur bacteria (PNSB) from the genus rhodopseudomonas or rhodospirillum. The microorganisms used in the composition and animal feed of the present invention may be a combination of different microorganisms such as a combination of bacteria from the genus rhodopseudomonas or rhodospirillum.

In another embodiment, the animal feed can be any form of food for animals including food for humans. The animal feed can be feed for mammals such as but not limited to pigs or for birds such as flamingoes and scarlets. In a preferred embodiment, the animal feed is an aquatic animal feed. The aquatic animal is a fish or a crustacean. Preferably, the aquatic animal is salmon or trout.

### BRIEF DESCRIPTION OF THE DRAWINGS

FIG. 1 shows the weight gain of fish over time after being fed feed containing the bacterial additive PNSB in Experiment 1. Table 1 (below) discloses the data obtained. As shown in FIG. 1 and Table 1, there is an increase in growth rate for those fish fed the bacterial additive.

TABLE 1
Salmon Evaluation
Test started on 18 October 2004
Fish Weights are in grams. Tank 1 is the control
and Tank 2 is the test tank. Weights are mean plus or minus SD.
At least thirty (30) fish per tank were weighted to give the
mean weights and the SD. Standard Deviation = SD

| DAY | Tank 1 | SD | Tank 2 | SD | difference | % |
|---|---|---|---|---|---|---|
| 0 | 345.7 | 27.39 | 345.5 | 25.89 | −0.2 | 0 |
| 35 | 419.9 | 55.57 | 437.7 | 48.44 | 17.8 | 4.24 |
| 92 | 481.7 | 80.8 | 526.7 | 94.8 | 45 | 9.34 |
| 128 | 533.7 | 93.71 | 595.7 | 121.33 | 61.96 | 11.6 |
| 168 | 619.7 | | 687.9 | | 68.2 | 11 |
| 211 | 695.6 | 216.4 | 824.2 | 234.6 | 128.55 | 18.5 |
| 239 | 803.8 | | 972.2 | | 168.4 | 20.95 |
| 270 | 950.1 | | 1078.3 | | 128.2 | 13.5 |
| 301 | 1078.3 | | 1150.2 | | 71.9 | 6.67 |

FIG. 2 shows the weight gain of fish over time after being fed feed containing the bacterial additive PNSB in Experiment 2. Table 2 (below) discloses the data obtained. As shown, there is an increase in growth rate for those fish fed the bacterial additive.

TABLE 2
Salmon Evaluation
Test started on 17 May 2005
Tank A: is the mean weight of the fish from the
two (2) control tanks taken together.
Tank B: is the mean weight of the fish from the
two (2) test tanks taken together.
Fifty (50) fish were weighed from each tank on the specified day.
Weight in grams.

| DAY | Tank A | Tank B | difference(gm) |
|---|---|---|---|
| 0 | 201.75 | 200.65 | −1.1 |
| 28 | 233.2 | 241.3 | 8.1 |
| 59 | 256.25 | 271.85 | 15.6 |
| 90 | 287.75 | 306.9 | 19.15 |

### DETAILED DESCRIPTION OF THE INVENTION

Carotenoids and Their Uses

Carotenoids is a generic name for a group of pigments having a chain of polyunsaturated hydrocarbons with 40 carbon atoms and having two terminal ring systems. Examples of carotenoids include, but are not limited to, β-carotene, astaxanthine, canthaxanthine, zeaxanthine, echinemone, adonirubin, donixanthine, lycopine, bixine, citranaxanthine, lutein, capsanthin, cryptoxanthine, β-apo-8′-carotenoic acid and its esters, β-apo-8′-carotenal, β-apo-12′-carotenal and mixtures thereof. Carotenoids composed entirely of carbon and hydrogen are known as carotenes such as β-carotene, while those that contain oxygen are xanthophylls such as astaxanthine.

Carotenoids play important roles in the growth and development as well as the survival of animals and plants. For example, carotenoids play a role in preventing cancer and in maintaining healthy vision. Specifically, xanthophylls function as chemo-protectives. Additionally, xanthophylls, such as adonirubin and astaxanthine, may also act as nutraceuticals that prevent carcinogenesis through anti-oxidative, anti-free radical, or other mechanisms. The beneficial nutraceutical functions of the carotenes and xanthophylls extend to the prevention of heart attacks and strokes.

Astaxanthine is a naturally occurring carotenoid, specifically a xanthophyll—in the same family of nutrients as vitamin A—and has a vital nutritional function as well as providing color to multicellular organisms. Astaxanthine is widely distributed in nature and is the predominant pigment in salmonids, shrimp, crab, lobster, and other crustaceans. Additionally, it produces the red coloration of some birds such as flamingos and the scarlet ibis (Weedon, B. C. L. (1971) Occurrence In: Carotenoids, O. Isler et al. (eds.), Halsted Press, New York, pp. 29-60).

One of the most important uses of the xanthophylls is in animal feed. Xanthophylls such as, but not limited to, astaxanthine and canthaxanthine are added to the diet of animals, such as farm raised fish which do not produce these pigments endogenously. Fish raised on fish farms and in hatcheries are white, and pale as contrasted with similar fish produced in their natural environment. These fish do not have the skin and flesh colors characteristic of fish produced in their natural environment. For this reason there is a strong consumer preference for fish taken from their natural environment, although nutritionally the farm produced fish may be identical to those produced in their natural environment. The pale color of fish raised on fish-farms or in hatcheries is improved when the fish are fed a diet supplemented by large quantities of dried, ground-up exoskeletal crustacean remains. However, development of a satisfactory color in this manner of feeding can only be achieved over long periods of time. It has been found that astaxanthine can be extracted from the exoskeletons of crustacean shells and tissues and fed, admixed with other feed in dietary formulations, to the farm fish in massive concentrations to develop satisfactory pigmentation over short periods of time. It also makes economical sense to be able to obtain the satisfactory color over a short period of time.

Thus, when fish such as, but not limited to, salmon, rainbow trout, red sea bream, or yellowtail are aquacultured, astaxanthine must be included as a dietary supplement in order to produce the coloration necessary for effective marketing (Torrissen, O. J. (1986) Aquaculture 53: 271-278). Astaxanthine remains one of the most expensive ingredients used in fish feed for farm raised fish.

Xanthophylls have also been investigated for the pigmentation of avian egg yolks because of the economic importance of color in chicken egg yolks. Yolks with high pigment content are more in demand. The most common pigment source in commercial diets has been yellow corn, which supplies the prominent egg yolk pigments cryptoxanthine, zeaxanthine and lutein. Unfortunately, corn in chicken diets is often replaced with higher energy grains such as milo, wheat, rice and barley, with the consequent loss in pigmentation in egg yolks. Xanthophylls, such as astaxanthine, may be used as a poultry food supplement to increase yolk pigmentation.

Chemical Production of Carotenoids

Carotenoids, specifically astaxanthine, can be produced chemically. For example, U.S. Pat. No. 4,245,109 provides a method of producing astaxanthine. Australian Patent 2003205699 and PCT Publication WO 03066583 provide a method of producing astaxanthine derivatives.

Recently, there has been some controversy surrounding the addition of chemical additives to fish feed, especially astaxanthine. Its allowable concentration in fish food, for example, has been reduced substantially in EU countries and there may even be an initiative to eliminate its use completely, especially in the USA. There are several reasons for these initiatives.

First of all chemically produced astaxanthine is a racemic mixture. It has several asymmetric carbons in its molecular structure which means that the chemically synthesized compound has both stereoisomers for each asymmetric carbon atom. On the other hand, a compound produced biologically or biochemically will have only one (1) of the stereoisomers, 3(S),3′(S)-astaxanthine or 3(R),3′(R)-astaxanthine. In addition, there are numerous “cis-trans” isomers possible due to the numerous carbon to carbon double bonds in the backbone structure of the carotenoid molecule. Biologically produced carotenoids are almost always in the “trans” configuration, in contrast to a chemically synthesized molecule which can have both “cis” and “trans” configurations. Furthermore, molecules produced biologically can be degraded biologically as well. That is not necessarily true for chemically produced molecules. Accordingly, there is an interest in using biologically produced instead of chemically produced carotenoids.

Microorganisms Producing Carotenoids

One way of using biologically produced carotenoids is to add them to the feed of animals. Thus far, biologically produced carotenoids have not been used as a food additive in fish food. The present invention provides supplementing microorganisms that produce carotenoids to animal feeds to promote the development of the desired color in animals and to increase the animals' growth rate. The microorganisms used in the present invention as food additive produce carotenoid pigments which impart the desired color in animals and/or increase the growth rate of animals. The animal may be a mammal, such as a pig; an avian such as a flamingo, a scarlet ibis, or a chicken; or an aquatic animal, such as the rainbow trout or salmon.

Carotenoids are found in animals, plants, microorganisms, and algae. Carotenoids can be produced not only chemically but also biosynthetically. Biological carotenoids can be produced recombinantly or naturally by microorganisms. U.S. Pat. No. 6,869,773 provides a method of producing carotenoids recombinantly from various microorganisms. U.S. Pat. No. 6,329,141 provides a transformed Phaffia strain that produces astaxanthine. Published U.S. Patent Application 20030077691 provides a recombinant means of producing astaxanthine using the genetic materials derived from Phaffia rhodozyma.

Bacteria that could be used to produce carotenoids naturally include but are not limited photosynthetic purple non-sulfur bacteria, heliobacteria, green non-sulfur bacteria, green sulfur bacteria, and purple bacteria. The carotenoids produced by these bacteria are similar, but their colors are different. For example, the heliobacteria produce neurosporene; the purple bacteria produce lycopine, spirilloxanthine, and okenone; the green non-sulfur bacteria produce β- and γ-carotenes; and the green sulfur bacteria produce chlorobactene, isorenieratene, and β-isorenieratene (Brock-Biology of Microorganisms, Edited by Madigan, Martinko and Parker, Published by Prentice-Hall International Inc., 1997, Chapter 16, pp 635-654).

The present invention uses microorganisms that produce carotenoids natively or recombinantly. Preferably, the microorganisms are the photosynthetic purple non-sulfur bacteria (PNSB). PNSB of the genera rhodopseudomonas and rhodospirillum produce carotenoid pigments as a part of their photosynthetic apparatus which are closely related to astaxanthine. These carotenoids are also highly colored, deep red compounds. Their structures are well known and identified in the bacterial literature. The present invention replaces chemically produced astaxanthine in the fish feed with similar compounds from bacteria which synthesize similar carotenoid compounds as a part of their photosynthetic apparatus. The present invention is useful, since these bacteria are totally natural, free-living species. In fact, the present invention provides the use of the entire bacterium as the additive rather than just the carotenoid pigments it produces.

Photosynthesis, by definition, is the use of light energy for the production of cellular components. The photosynthetic apparatus of the cell captures light energy chemically and uses that energy for its synthetic needs. The bacteria are distinct from green plants and algae in that they cannot split water during their photosynthesis and produce oxygen as do the plants and green algae. In addition, the bacteria grow on organic material like neutralized vinegar or yeast extract rather than carbon dioxide like the green plants.

The bacteria grown anoxically and containing high levels of bacterial colored carotenoid compounds can be used as a food additive in animal feed. The bacteria may be added to the feed of animals, for example, mammals such as pigs. The bacteria could also be used to feed avians such as flamingos who get their pink color from brine shrimp when living in the wild and chickens who may benefit from the bacterial carotenoids in deeper yellow yolks in their eggs. Preferably, the bacteria are added to the feed of aquatic animals such as, but not limited, to fish including salmon, trout, and koi (an ornamental highly colored carp revered by the Japanese and worth thousands of dollars in some cases).

It is also possible to grow the bacteria aerobically, that is in the presence of oxygen, to obtain biomass to be used as a food additive for animals other than those for which colored flesh is desired. This would include a food additive for man as distasteful as that may sound. There are three primary carbohydrate based foods eaten throughout the world. Bacteria or bacterial protein can be added to any of them. Bacteria can be grown very quickly and turned into a powder for easy storage, transport and incorporation into any base, for example, manioc (cassava), potato, or rice. The extremely rapid growth rate of the bacteria makes them an ideal source of inexpensive protein which will satisfy all of the amino acid nutritional needs of any animal including humans. Single cell protein is valuable and inexpensive. All the benefits of the bacterial mass accrue with aerobically grown cells except the presence of the colored bacterial carotenoid compounds. The present invention uses bacteria that produce carotenoids as a food additive for animals including humans.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure.

### EXAMPLES

### Example 1

### Aquatic Animals

Methods

Photosynthetic bacteria of the genus rhodopseudomonas were grown under anoxic conditions and constant illumination from CW fluorescent lamps. They were harvested by centrifugation to obtain a paste similar to butter. The bacteria were added to a standard fish feed formula which did not contain astaxanthine and fed to trout for a period of about eight (8) weeks. The level of bacterial addition to the fish feed was about 11% by weight. A control group of trout was also included using the same feed without the bacteria added to it. After the eight week period, only the fish fed the feed containing the photosynthetic bacteria had flesh was pink in color. This corroborates the idea that photosynthetic bacteria containing these colored carotenoid pigments can substitute entirely for astaxanthine as the means to color the fish flesh pink and simulate the color of flesh from fish raised in the wild.

The PNSB produce high levels of bacterial carotenoid pigments, a part of their photosynthetic apparatus, needed for the development of the pink color in the flesh of the fish only when growing in the absence of oxygen i.e. anoxically. In the presence of oxygen the production of the carotenoid pigments is prevented. So for use as the bacterial fish food additive used to color the flesh of the salmon, trout, or koi, the bacteria must be grown under conditions of anoxia.

In the experiments that were performed with salmon (as described in detail below), the amount of bacteria added was not more than about 500 grams to about 25 kilograms of food, much less than was used in the experiments with the trout. This amounts to about 2% of the food mass. In some cases, the amount was less than 2%. Generally about 2% of the weight of the food as a suspension of 20% bacterial mass, w/v, in water and/or propylene glycol was used to coat the fish feed. These bacteria were grown on defined media, not waste material, harvested and resuspended in the water and/or propylene glycol to which is added 1% sodium thiosulfate as a preservative. This suspension was used to coat the fish food. Coating the feed in this manner precluded using larger quantities of the bacterial suspension per unit weight of feed as was done with trout.

The bacteria were grown on defined media. There was no need to pasteurize our bacteria; in fact, pasteurization would kill the majority of them and eliminate two (2) of the specific advantages of live bacteria in the gut of the host animal, that of improved food conversion and the other, early waste processing. In addition, by using a defined media, it can be sterilized to prevent unwanted organisms initially rather than having to pasteurize the suspension later. The bacteria are added to or coated on the fish food. They do not serve as the only food source for the fish.

Anoxic conditions were created by having the culture media stand undisturbed in a cylindrical tank containing the bacterial inoculum for about an hour. The PNSB can grow aerobically in the dark using oxygen for their growth. Then the illumination can be provided by fluorescent, incandescent or high pressure sodium vapor lamps. Fluorescent lamps were used in these studies to avoid the problem of heat generation as both incandescent and high pressure sodium vapor lamps get very hot when providing constant illumination. The nutrient media under constant illumination was stirred slowly once or twice during the ten (10) day growth period. During this process, it is desirable not to introduce oxygen into the growth tank.

The growth tanks for the initial work were cylindrical in shape having the dimensions of radius (R) 35 cms and height (H) 85 cms for a volume of 325 liters. The tank was totally enclosed and had an access cover to allow additions and occasional stirring. Such a tank will provide anoxic conditions within an hour or so of start-up without further manipulation. The PNSB themselves will scavenge any residual oxygen themselves as they prefer to grow aerobically. The purple sulfur bacteria like Chromatium will not do that as they only grow anoxically and in the presence of light.

A plexiglass cylinder within the growth tank to house the lamps providing the illumination for photosynthetic growth can be used for commercial production. This cylinder will house high pressure sodium vapor lamps and a system of ventilation around the lamps for cooling. Thus one can have external illumination or internal Illumination for the process of bacterial growth. Such a tank would be a cylindrical structure of R equal to two (2) meters and H equal to eight (8) meters having a volume of 100 cubic meters with internal continuous illumination.

The bacteria used in these experiments were living organisms. The carotenoids were not extracted from the bacteria and used as one might a chemical additive. There were several reasons for conducting the trials as described. First, the bacteria are about 80% protein by weight and, being alive, have certain metabolic capabilities. They can begin to process the fish feed while it is in the digestive tract of the fish and thereby increase the food conversion ratio for that feed. Thus, less feed is required to produce the same weight of fish in the presence of the bacteria than in their absence. They are an excellent fish food in and of themselves. The bacterial protein provides all the necessary amino acids for the production of fish protein. Second, the bacteria can also begin the process of digestion of the fish waste while it is still present in the intestinal tract of the fish thereby decreasing the amount of waste excreted by the fish during their growth cycle. Each of these capabilities of the bacteria is a monetary advantage to the fish farmer, as well as an advantage for the environment in which the fish are raised.

Experiments with Salmon

The methods described above were modified for salmon. Briefly, the quantities of material used for feed preparation and the composition of the bacterial suspension varied from batch to batch, but the procedure was generally as follows:
The bacterial preparation (PNSB) was mixed with propylene glycol (PG) and fish oil (Cod Liver Oil) using an electric blender. Commercial fish feed pellets were evenly coated with this mixture using quantities of approximately 1 kg liquid to 10 kg feed pellets. Liquid was slowly added to the feed while it was being turned in a cement mixer. Coated pellets were stored in plastic bags and left overnight before use for liquid containing the bacteria to be absorbed into the pellets.
   Ole J. Torrissen provides detailed procedures and evaluation techniques for assessing carotenoids both in the flesh and serum of fish (Carotenoid Pigmentation of Salmonids by Ole J. Torrissen, Dissertation for the Degree of Doctor of Philosophiae, Institute of Marine Research, Matre Aquaculture Station, 5198 Matredal, Norway and Department of Fisheries Biology, University of Bergen, Bergen, Norway 1989).

The first set of experiments with salmon, labeled as Experiment 1, was started in October 2004, lasted approximately (10) months and was conducted with varying amounts of photosynthetic bacteria of genus rhodopseudomonas (referred to in the Tables as PNSB) added to the feed for approximately fifty (50) fish in the test batch which were evaluated against the same number of fish in a control tank. The results showed a consistent increase in weight gain (food conversion ratio) in the test batch versus the control batch over the period of the evaluation. While the test nutrient mix had plenty of carotenoid present in it, none was transferred to the fish serum or flesh. Thus, the weight gain was mitigated by the bacteria themselves and the carotenoid had no effect.

A second set of experiments, Experiment 2, began in June 2005 with the intent to dispel any tank effect in the first test set and look at the effect of rupturing the bacterial cell wall to release the carotenoid pigment from the cells. Four (4) tanks of fifty (50) fish each were used. Bacterial cells were ruptured using lysozyme enzyme from egg white together with sonnication. Two (2) tanks served as test tanks and two (2) tanks were used as controls. The results showed again that a consistent weight gain difference took place in the test tanks versus the control tanks and no tank effect was observed. Furthermore, pigment uptake could be observed in the flesh of the fish after two (2) months albeit only slightly. The results of pigment uptake in the serum were similar to those for the flesh. These results are important as they show that astaxanthine itself may not be the only carotenoid that can provide the rose color to the salmon flesh and that a biologically produced xanthophyll molecule will suffice. Additionally, the weight gain or food conversion ratio increases followed the same pattern with this test as those seen in the data from the first experiment. One may conclude that weight gain increases can be provided with the bacterial cells devoid of the carotenoid but it is essential for pigment deposition in the flesh of the fish.

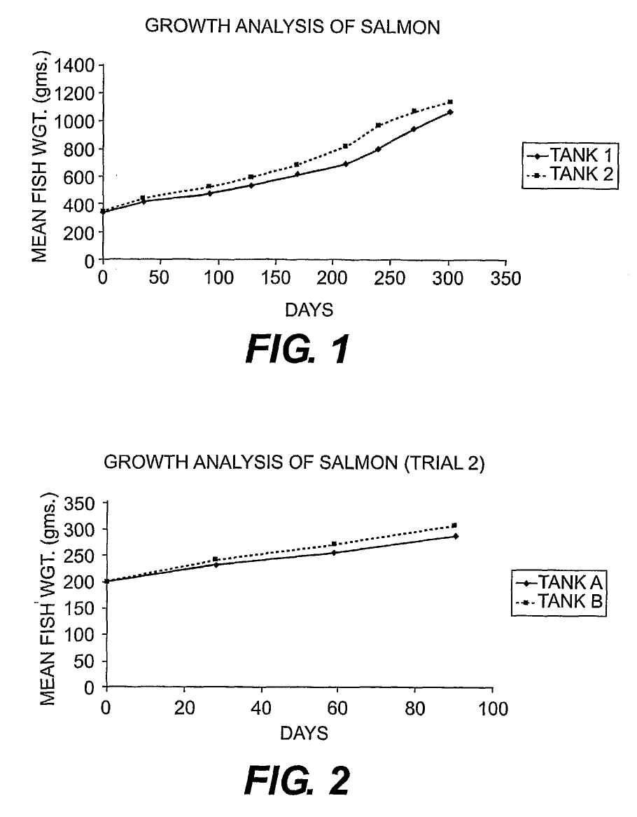

Tables 3 and 4 (below) summarize the results of the Experiments 1 and 2, respectively, as do FIGS. 1 and 2 which show the increase in weight of the fish after being fed feed containing bacterial additive PNSB.

TABLE 3
Results of Experiment 1
1. Feed composition

| | | | PG/water | oil | feed | | total carotenoid concentration |
|---|---|---|---|---|---|---|---|
| date | diet | PNSB (g) | (g) | (g) | (kg) | PNSB/feed | in feed (ppm) |
| Oct. 13, 2004 | control | 0 | 1000 | 1000 | 25 | 0 | 16.9 |
| | A1 | | | | | | |
| | test A1 | 1000 | 0 | 1000 | 25 | 40 | 25.3 |
| Jan. 18, 2005 | control | 0 | 200 | 800 | 10 | 0 | 16.3 |
| | A2 | | | | | | |
| | test A2 | 850 | 0 | 150 | 10 | 85 | 48.5 |
| Feb. 23, 2005 | control | 0 | 200 | 900 | 11 | 0 | |
| | A3 | | | | | | |
| | test A3 | 560 | 100 | 440 | 11 | 51 | |
| Jun. 13, 2005 | control | 0 | 180 | 620 | 8 | 0 | |
| | A4 | | | | | | |
| | test A4 | 385 | | 415 | 8 | 48 | |
| Jul. 14, 2005 | control | 0 | 285 | 800 | 12 | 0 | |
| | A5 | | | | | | |
| | test A5 | 280 | 0 | 900 | 12 | 23 | |

2. Mean weight (g)

| Date | Control | PNSB | PNSB/control | t-value | p | |
|---|---|---|---|---|---|---|
| Oct. 18, 2004 | 345.7 | 345.5 | 1.00 | 0.0483 | 0.9616 | not significant |
| Nov. 22, 2004 | 419.9 | 437.7 | 1.04 | 1.3 | 0.1987 | not significant |
| Jan. 18, 2005 | 481.7 | 526.7 | 1.09 | 1.944 | 0.0567 | not significant |
| Feb. 23, 2005 | 533.7 | 595.7 | 1.12 | 2.311 | 0.024 | p < 0.05 |
| Apr. 4, 2005 | 619.7 | 687.9 | 1.11 | 1.698 | 0.0948 | not significant |
| May 17, 2005 | 695.6 | 824.2 | 1.18 | 2.483 | 0.0153 | p < 0.05 |
| Jun. 14, 2005 | 803.8 | 972.2 | 1.21 | 2.564 | 0.0124 | p < 0.05 |
| Jul. 15, 2005 | 950.1 | 1078.3 | 1.13 | 2.161 | 0.0339 | p < 0.05 |
| Aug. 15, 2005 | 1078.3 | 1150.2 | 1.07 | 1.604 | 0.1129 | not significant |

3. Average growth rate since start of experiment (% body weight per day)

| date | Control | PNSB | PNSB/control |
|---|---|---|---|
| Nov. 22, 2004 | 0.56 | 0.68 | 122% |
| Jan. 18, 2005 | 0.36 | 0.46 | 127% |
| Feb. 23, 2005 | 0.34 | 0.43 | 125% |
| Apr. 4, 2005 | 0.35 | 0.41 | 118% |
| May 17, 2005 | 0.33 | 0.41 | 124% |
| Jun. 14, 2005 | 0.35 | 0.43 | 123% |
| Jul. 15, 2005 | 0.37 | 0.42 | 113% |
| Aug. 15, 2005 | 0.38 | 0.40 | 106% |

4. Average apparent feed conversion rate since start of experiment

| date | Control | PNSB | PNSB/control |
|---|---|---|---|
| Nov. 22, 2004 | 1.87 | 1.50 | 80% |
| Jan. 18, 2005 | 2.29 | 1.72 | 75% |
| Feb. 23, 2005 | 2.23 | 1.68 | 75% |
| Apr. 4, 2005 | 2.06 | 1.66 | 81% |
| May 17, 2005 | 2.12 | 1.59 | 75% |
| Jun. 14, 2005 | 2.05 | 1.53 | 75% |
| Jul. 15, 2005 | 1.91 | 1.61 | 84% |
| Aug. 15, 2005 | 1.94 | 1.76 | 90% |

5. Flesh pigment

| date | Control | PNSB | PNSB/control |
|---|---|---|---|
| Nov. 22, 2004 | 1.02 | 1.05 | 103% |

PNSB = wt of bacterial suspension as supplied
Figures are total pigment (ppm) measured by spectrophotometer in extracts from flesh samples from 3 fish per tank.
Spectrophotometer readings were taken at 470 nm.

TABLE 4
Results of Experiment 2
1. Feed composition

| | | | | | | | total carotenoid concentration |
|---|---|---|---|---|---|---|---|
| date | diet | PNSB (g) | water (g) | PG (g) | oil (g) | feed (kg) | in feed (ppm) |
| May 11, 2005 | control B1 | 0 | 150 | 300 | 560 | 6.5 | not determined |
| | test B1 | 150 | 150 | 150 | 560 | 6 | not determined |
| Jun. 22, 2005 | control B2 | 0 | 0 | 380 | 620 | 10 | 5.71 |
| | test B2 | 380 | 0 | 300 | 620 | 10 | 12.26 |

In batch test B1, PNSB was treated with lysozyme to crack bacterial cells before incorporation into feed.
In feed batch test B2, PNSB was treated with lysozyme and sonicated to crack bacterial cells before incorporation into
feed.

2. Mean weight (g)

| | Control | | | PNSB | | | | Statistical comparison of control | | |
|---|---|---|---|---|---|---|---|---|---|---|
| Date | tank | tank | | tank | tank | | | vs PNSB | | |
| dd-mm-yy | rep 1 | rep 2 | mean | rep 1 | rep 2 | mean | PNSB/control | t-value | p | |
| May 17, 2005 | 201.3 | 202.2 | 201.75 | 200.6 | 200.7 | 200.65 | 99% | 0.2823 | 0.0778 | not |
| | | | | | | | | | | significant |
| Jun. 14, 2005 | 229.7 | 236.7 | 233.2 | 238.7 | 243.9 | 241.3 | 103% | 1.66 | 0.0985 | not |
| | | | | | | | | | | significant |
| Jul. 15, 2005 | 253.4 | 259.1 | 256.25 | 272.3 | 271.4 | 271.85 | 106% | 2.498 | 0.0133 | <0.05 |
| Aug. 15, 2005 | 279.9 | 287.6 | 283.75 | 307.4 | 306.4 | 306.9 | 108% | 2.965 | 0.0034 | <0.01 |

Figures are mean weight (g) of all fish in each tank (n ~ 50 per tank).

3. Average growth rate since start of experiment (% body weight per day)

| date | Control | PNSB | PNSB/control |
|---|---|---|---|
| Jun. 14, 2005 | 0.52 | 0.66 | 127% |
| Jul. 15, 2005 | 0.41 | 0.51 | 127% |
| Aug. 15, 2005 | 0.38 | 0.47 | 125% |

4. Average apparent feed conversion rate since start of experiment

| dd-mm-yyyy | Control | PNSB | PNSB/control |
|---|---|---|---|
| Jun. 14, 2005 | 1.86 | 1.30 | 70% |
| Jul. 15, 2005 | 2.38 | 1.74 | 73% |
| Aug. 15, 2005 | 2.31 | 1.71 | 74% |

5. Serum pigment

| | | | | PNSB | | | |
|---|---|---|---|---|---|---|---|
| | control | tank rep | | tank rep | tank rep | | |
| date | tank rep 1 | 2 | average | 1 | 2 | average | PNSB/control |
| Jun. 14, 2005 | | | 0 | | | 0.07 | |
| Jul. 13, 2005 | 0.185 | 0.127 | 0.156 | 0.154 | 0.129 | 0.1415 | 0.91 |
| Aug. 15, 2005 | 0.201 | 0.225 | 0.213 | 0.275 | 0.3 | 0.2875 | 1.35 |

Figures are absorbance of serum measured at 470 nm relative to distilled water blank in pooled serum
samples from 3 fish per tank, except Jun. 14, 2005 which shows absorbance of pooled test samples relative to
pooled control samples.

6. Flesh pigment

| | control | tank rep | | PNSB | tank rep | | |
|---|---|---|---|---|---|---|---|
| date | tank rep 1 | 2 | average | tank rep 1 | 2 | average | PNSB/control |
| Aug. 15, 2005 | 1.38 | 1.14 | 1.26 | 1.26 | 1.99 | 1.625 | 1.29 |

Figures are total pigment (ppm) measured by spectrophotometer in extracts from pooled flesh samples from
3 fish per tank. Spectrophotometer readings were taken at 470 nm.

In these experiments, the bacteria added to the fish feed of farm raised fish replaced chemically produced astaxanthine normally used in the diets of these fish. Tables 3 and 4 and FIGS. 1 and 2 indicate that farm raised fish fed with the bacteria acquired not only the desired pigmentation but also an increase in growth rate as compared to those fed with their normal food. The weight gain data is shown under “mean weight” in both tables. The pigmentation in the flesh is 29% greater in the test fish than the control fish as shown in “flesh pigment” in Table 4. This is corroborated by the 35% increase in “serum pigment” in the same experiment.

Since the amount of bacteria is about 2% or less of the food mass, it is not possible for the weight gains noted in the Tables and Figures to be attributed to the mass of bacteria added. The increase in growth rate could be due to the bacteria pre-digesting some of the food that was eaten, stimulating the fish to eat more of the food and/or having an effect on the growth hormone of the fish. The maximum difference during the first experiment was 21% or 168 grams per fish as shown in Table 1, “mean weight for 14 Jun. 2005.” Since there were only thirty four (34) fish left in the tank by this time, this is about six (6) kgs difference in total weight. Additionally, toward the end of the first experiment, the weight difference declined. When these fish reached a weight of approximately 1 kg, they started to mature, that is develop gonads. Much of their growth energy goes into the development of gonads and the test fish reached that level of growth faster than the control fish, hence the decline in the weight difference. Commercially raised fish are harvested before gonad development starts just to preclude the loss in growth to gonad development.

When trout, a fresh water fish, was tested, the color was imparted to the flesh using whole bacteria. Such was not the case with the salmon. Similar growth rates in salmon were noted with both whole and ruptured bacteria. But the uptake of color, carotenoid, was not observed in the salmon until ruptured bacteria were added to the feed. The technique of rupturing the bacteria did not disintegrate more than 50% of the bacteria cells. The growth rates seen were practically identical when whole bacterial addition was compared to ruptured bacterial addition.

The pigmentation and growth rate changes mitigated by the bacteria are not specific to salmon and trout, but are observable in other farm raised fish, such as cod and halibut and other farm raised fish that grow in cold waters, and may also be observable in farm raised fish that grow in warm waters such as the Mediterranean Sea, and in animals such as mammals and avians, for example pigs, chickens, and flamingoes.

### Example 2

### Non-Aquatic Animals

The conditions described above for preparing the bacteria as an additive to feed of aquatic animals are applicable to preparing the bacteria as supplement to the food of other animals including humans. The conditions may be modified by one of ordinary skill in the art to suit the needs of other animals. For example, the bacterial product may be used to only increase the growth rate of animals. In this case, the same bacteria (PNSB) may be grown aerobically in the dark. Therefore, the energy for growth is provided by oxygen not light.

The method of harvesting the bacteria is also the same for the products grown under anoxic or aerobic conditions. The only exception to this would be the preparation of a bacterial powder for addition to carbohydrate foods such as manioc (cassava or tapioca), potatoes and rice. Here there would be an additional step of drying the bacteria without destroying the protein contained within them. The product which results during the preparation of the bacteria for the addition to the fish feed, for example, is a paste having the consistency of cold margarine. That product would not be suitable for addition to a carbohydrate based powder for shipment throughout the world. Thus the need for the additional step of drying the bacteria.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference in their entirety.

1. A composition comprising photosynthetic purple non-sulfur bacteria (PNSB) and animal feed.

2. A composition of claim 1, wherein the PNSB are from the genus rhodopseudomonas or rhodospirillum.

3. An animal feed comprising photosynthetic purple non-sulfur bacteria (PNSB).

4. An animal feed of claim 3, wherein the photosynthetic bacteria is from the genus rhodopseudomonas or rhodospirillum.

5. A method of making an animal feed of claim 3 comprising growing the PNSB, harvesting the bacteria, and adding the bacteria to an aquatic animal feed.

6. A method of claim 5, wherein the PNSB are grown under anoxic conditions and constant illumination.

7. A method of claim 5, wherein the PNSB are from the genus rhodopseudomonas or rhodospirillum.

8. A method of cultivating an animal to develop a desired pigment comprising,
a) obtaining an animal feed supplemented with an effective amount of PNSB, wherein the feed is not supplemented with astaxanthine;
b) administering the supplemented feed to the animal; and
c) cultivating the animal to grow under conditions that allow development of the pigment.

9. A method of increasing the growth rate of an animal comprising,
a) obtaining an animal feed supplemented with an effective amount of PNSB, wherein the feed is not supplemented with astaxanthine;
b) administering the supplemented feed to the animal; and
c) cultivating the animal to grow under conditions that allow the animal to grow.

10. A method of decreasing the amount of waste excretion by an animal comprising
a) obtaining an animal feed supplemented with an effective amount of PNSB, wherein the feed is not supplemented with astaxanthine;
b) administering the supplemented feed to the animal; and
c) cultivating the animal to grow under conditions that allow digestion of animal waste while still present in the intestinal tract of the animal thereby decreasing the amount of waste excreted by the animal during growth.

11. A method of claim 8, wherein the bacteria are from the genus rhodopseudomonas or rhodospirillum.

12. A method of claim 8, wherein the animal is an aquatic animal.

13. A method of claim 12, wherein the aquatic animal is a fish or a crustacean.

14. A method of claim 13, wherein the fish is a salmon or trout.

15. A method of claim 13, wherein the crustacean is a lobster, shrimp, or crab.

16. A composition of claim 2, wherein the animal feed is an aquatic animal feed.

17. An animal feed of claim 4, wherein the animal feed is an aquatic animal feed.

18. A method of claim 7, wherein the animal feed is an aquatic animal feed.

19. A method of claim 8, wherein the PNSB are grown under anoxic conditions before supplementing the animal feed.

20. The method of claim 9, wherein the PNSB are grown aerobically before supplementing the animal feed.

21. The method of claim 8, wherein the PNSB are produced recombinantly.

22. The composition of claim 2, wherein the PNSB are grown under anoxic conditions.

23. The composition of claim 2, wherein the PNSB are grown under aerobic conditions.

24. The composition of claim 2, wherein the PNSB are produced recombinantly.

25. The animal feed of claim 4, wherein the PNSB are grown under anoxic conditions.

26. The animal feed of claim 4, wherein the PNSB are grown under aerobic conditions.

27. The animal feed of claim 4, wherein the PNSB is produced recombinantly.
